(19) United States
(12) Patent Application Publication
(10) Pub. No.: US 2024/0324537 A1
(43) Pub. Date: Oct. 3, 2024

(54) Raspberry Plants

(71) Applicant: BBR IP B.V., Breda (NL)

(72) Inventors: Johannes Adrianus Petrus de Jongh, Breda (NL); Johannes Jacobus Maria Lambalk, Breda (NL)

(21) Appl. No.: 18/576,164

(22) PCT Filed: Jul. 6, 2021
(86) PCT No.: PCT/EP2021/068669

§ 371 Date: Jan. 3, 2024

(51) Int. Cl. A01H 6/74; A01H 5/08

(52) U.S. Cl. CPC A01H 6/7499; A01H 5/08

(57) ABSTRACT

Provided herein are improved raspberry plants, methods for providing the present improved raspberry plants and the use of present improved raspberry plant for providing improved plant vigour, higher fruit yield, more and better resistances to diseases and the prevention or absence of root suckers during cultivation. Specifically, provided herein are improved raspberry plants including a raspberry scion grafted on a blackberry rootstock. Specifically, disclosed herein are raspberry plants including a raspberry scion grafted on a blackberry rootstock.

### CROSS-REFERENCE TO RELATED APPLICATION

This application is the United States national phase of International Application No. PCT/EP2021/068669 filed Jul. 6, 2021, the disclosure of which is hereby incorporated by reference in its entirety.

### BACKGROUND OF THE INVENTION

### Field of the Invention

The present invention relates to improved raspberry plants, methods for providing the present improved raspberry plants and the use of present improved raspberry plants for providing improved plant vigour, higher fruit yield, more and better resistances to diseases and the absence or prevention of root suckers during cultivation.

### Description of Related Art

Raspberries are an important commercial fruit crop grown in all temperate regions of the world. Many of the most important commercial red raspberry cultivars are derived from hybrids between Rubus idaeus and Rubus strigosus. Cultivars that contain improved characteristics are important for the fresh market.

Raspberries have also been crossed with various species in other subgenera of the genus Rubus resulting in a number of hybrids, the first of which was the loganberry. Later notable hybrids include olallieberry, boysenberry, marionberry, and tayberry; all being multi-generational hybrids.

Hybridization between cultivated red raspberries and a few Asiatic species of Rubus has also been achieved.

Blackberry is an edible fruit produced by many species in the genus Rubus in the family Rosaceae, hybrids among these species within the genus Rubus, and hybrids between Rubus and Idaeobatus.

What distinguishes blackberry from its raspberry relatives is whether or not the torus (receptacle or stem) “picks with” (i.e., stays with) the fruit. When picking a blackberry fruit, the torus stays with the fruit. With a raspberry, the torus remains on the plant, leaving a hollow core in the raspberry fruit.

The term bramble, a word meaning any impenetrable thicket, has traditionally been used to designate blackberries. The usually black fruit is not a berry in the botanical sense of the word.

Botanically it is termed an aggregate fruit, composed of small drupelets.

Unmanaged mature blackberry plants form a tangle of dense arching stems, the branches rooting from the node tip on many species when they reach the ground. Vigorous and growing rapidly in woods, scrub, hillsides, and hedgerows, blackberry shrubs tolerate poor soils, readily colonizing wasteland, ditches, and vacant lots.

### SUMMARY OF THE INVENTION

It is an object of the present invention, amongst other objects, to provide raspberry plants with improved characteristic such as improved plant vigour, higher fruit yield, more and better resistances to diseases and the absence or prevention of root suckers during cultivation.

This object, amongst other objects, is met by the present invention by providing plants as outlined in the appended claims.

Specifically, this object, amongst other objects, is met by the present invention by providing improved raspberry plants comprised of a raspberry scion grafted on a blackberry rootstock.

Grafting, or graftage, is a horticultural technique whereby tissues of plants are joined so as to continue their growth together. The upper part of the combined plant is generally designated as the scion while the lower part is generally designated as the rootstock. The success of this joining requires that the vascular tissues grow together and such joining is called inosculation. The technique is most commonly used in asexual propagation of commercially grown plants for the horticultural and agricultural trades. According to the present invention, a blackberry plant is selected for its roots and a raspberry plant is selected for its stems and fruits.

The present inventors have surprisingly found that a blackberry rootstock provides a number of beneficial characteristics to a raspberry plant as compared to its “own” root system. It has been observed that raspberries with a blackberry root system exhibit improved plant vigour, higher fruit yield, more and better resistances to diseases and the absence or prevention of root suckers during cultivation.

According to a preferred embodiment, the present improved raspberry plants have a blackberry rootstock is selected from the group consisting of rootstocks from the blackberry species Rubus ursinus, Rubus laciniatus, Rubus argutus, Rubus armeniacus, Rubus plicatus, Rubus ulmifolius, Rubus allegheniensis, Rubus caesius, Rubus gratius, Rubus odoratus, Rubus spectabilis, Rubus stereobelus, Rubus corylifoii spec. (x dumetorum), Rubus sulcatus, Rubus foliosus, Rubus prei, Rubus pedemontanus, Rubus rudis, Rubus oreades, Rubus rosaceus, Rubus derasifolius, Rubus flexuosus, Rubus gratus, Rubus dejonghii, Rubus nemorosus, Rubus glareosus, Rubus geniculatus, Rubus diversus, Rubus lesdainii, Rubus drymophilus, Rubus polyanthemus, Rubus vestitus, Rubus rufescens, Rubus holerythos, Rubus macrophyllus, Rubus ulmifolius, Rubus armeniacus, Rubus nitidiformis, Rubus nemoralis laciniatus, Rubus insectifolius, Rubus subinermoides, Rubus platyacanthus, Rubus vigorosus, Rubus nessensis, Rubus praticolor, Rubus arcticus and intra/inter specific hybrids thereof.

Below, the above blackberry species are, if available, identified by their common Dutch/Belgian designations: Rubus corylifolii spec. (x dumetorum): hazelaarbraam or straatbraam; Rubus sulcatus: groefstokbraam; Rubus caesius: dauwbraam; Rubusfoliosus: bladhumusbraam or loofrijke braam; Rubus prei: fijntandige kambraam or fijntandige braam; Rubus pedemontanus: sierlijke woudbraam or armbloemige braam; Rubus rudis: ruwe raspbraam or ruwlootbraam; Rubus oreades: nimfwoudbraam or donkere braam; Rubus rosaceus: rode borstelbraam or fijnklierige braam; Rubus derasifolius: breedbladige humusbraam or breedbladige braam; Rubus flexuosus: slanghumusbraam or zigzagbraam; Rubus gratus: zoete haarbraam or zandbraam; Rubus dejonghii: bleke contrastbraam or vergeten braam; Rubus nemorosus: brede randbraam or roze roosjesbraam; Rubus glareosus: kiezelhumusbraam or slanke braam; Rubus geniculatus: knieviltbraam or knikbraam; Rubus diversus: late muisbraam or heggenbraam; Rubus lesdainii: brakelse haagbraam or brakelse braam; Rubus drymophilus: dichtbloemige humusbraam or dichtbloemige braam; Rubus polyanthemus: rijkbloemige haagbraam or rijkbloemige braam; Rubus vestitus: fraaie kambraam or kleine leembraam; Rubus rufescens: rosse humusbraam or tederroze braam; Rubus holerythos: prachtkoepelbraam or fraaibloemige braam; Rubus plicatus (yn. fruticosus): geplooide stokbraam or grillige braam; Rubus macrophyllus: bolle haarbraam or bolbladige braam; Rubus ulmifolius: koebraam; Rubus armeniacus: dijkviltbraam or armeense braam; Rubus nitidiformis: agressieve koepelbraam or agressieve braam; Rubus stereobelus: grote leemhaagbraam or grote leembraam; Rubus nemoralis laciniatus: peterseliehaagbraam or slipbladige braam; Rubus insectifolius: ingesneden humusbraam or veellootbraam; Rubus subinermoides: belgische haarbraam or belgische braam; Rubus platyacanthus: platte haagbraam or diklootbraam; Rubus vigorosus: zeegroene koepelbraam or zeegroene braam; Rubus nessensis: vroege roggebraam or vroege braam; Rubus praticolor: weide woudbraam or smalbladige braam.

According to yet another preferred embodiment, the present raspberry scion is selected from raspberry species selected from the group consisting of Rubus crataegifolius, Rubus gunnianus, Rubus idaeus, Rubus leucodermis, Rubus occidentalis, Rubus parvifolius, Rubus phoenicolasius, Rubus rosifolius, Rubus strigosus, Rubus ellipticus and intra/inter specific hybrids thereof, preferably Rubus idaeus and intra/inter specific hybrids thereof.

Below, the above raspberry species are, if available, identified by their common designations Rubus crataegifolius: Asian raspberry; Rubus gunnianus: Tasmanian alpine raspberry; Rubus idaeus: red raspberry or European red raspberry; Rubus leucodermis: whitebark raspberry or western raspberry, blue raspberry, black raspberry; Rubus occidentalis: black raspberry; Rubus parvifolius: Australian native raspberry; Rubus phoenicolasius: wine raspberry or wineberry; Rubus rosifolius: Mauritius raspberry; Rubus strigosus: American red raspberry (syn. R. idaeus var. strigosus); Rubus ellipticus: yellow Himalayan raspberry.

The present invention also relates to methods for providing the present improved raspberries, the methods comprise the steps of:
a) providing a blackberry rootstock;
b) grafting a raspberry scion on the blackberry rootstock;
c) allowing the graft to develop into improved raspberry plant.

One or more factors influencing a successful graft which can be considered in the present methods are:

Compatibility of scion and rootstock: Because grafting involves the joining of vascular tissues between the scion and rootstock, plants lacking vascular cambium, such as monocots, cannot normally be grafted. As a general rule, the closer two plants are genetically, the more likely the graft union will form. Genetically identical clones and intra-species plants have a high success rate for grafting. Grafting between species of the same genus is sometimes successful. Grafting has a low success rate when performed with plants in the same family but in different genera and grafting between different families is rare.

Cambium alignment and pressure: The vascular cambium of the scion and rootstock should be tightly pressed together and oriented in the direction of normal growth. Proper alignment and pressure encourages the tissues to join quickly, allowing nutrients and water to transfer from the rootstock to the scion.

Completed during appropriate stages of plant growth: The grafting is completed at a time when the scion and rootstock are capable of producing callus and other wound-response tissues. Generally, grafting is performed when the scion is dormant, as premature budding can drain the grafting site of moisture before the grafting union is properly established. Temperature greatly affects the physiological stages of plants. If the temperature is too warm, premature budding may result. Elsewise, high temperatures can slow or halt callus formation.

Proper care of graft site: After grafting, it is important to nurse the grafted plant back to health for a period of time. Various grafting tapes and waxes can optionally be used to protect the scion and rootstock from excessive water loss. Furthermore, depending on the type of graft, twine or string is used to add structural support to the grafting site. Sometimes it is necessary to prune the site, as the rootstock may produce shoots that inhibit the growth of the scion.

Suitable grafting techniques according to the present invention are cleft grafting, whip grafting, stub grafting, four-flap grafting, awl grafting, veneer grafting and rind grafting.

In cleft grafting a small cut is made in the rootstock and then the pointed end of the scion is inserted in the rootstock. This is best done in the early spring and is useful for joining a thin scion about 1 cm diameter to a thicker branch or rootstock. It is best if the former has 3-5 buds and the latter is 2-7 cm in diameter. The branch or rootstock should be split carefully down the middle to form a cleft about 3 cm deep. If it is a branch that is not vertical then the cleft should be cut horizontally. The end of the scion should be cut cleanly to a long shallow wedge, preferably with a single cut for each wedge surface, and not whittled. A third cut may be made across the end of the wedge to make it straight across.

The wedge is slid into the cleft so that it is at the edge of the rootstock and the centre of the wedge faces are against the cambium layer between the bark and the wood. The top of the rootstock is taped to hold the scion in place and covered with grafting wax or sealing compound. This stops the cambium layers from drying out and also prevents the ingress of water into the cleft.

In whip grafting, the scion and the rootstock are cut slanting and then joined. The grafted point is then bound with tape and covered with a soft sealant to prevent dehydration and infection by germs. The common variation is a whip and tongue graft, which is considered the most difficult but has the highest rate of success as it offers the most cambium contact between the scion and the rootstock. It is the most common graft used in preparing commercial fruit trees. It is generally used with stem less than 1.25 cm diameter, with the ideal diameter closer to 1 cm and the scion should be of roughly the same diameter as the rootstock.

The rootstock is cut through on one side only at a shallow angle with a sharp knife. The scion is similarly sliced through at an equal angle starting just below a bud, so that the bud is at the top of the cut and on the other side than the cut face.

In the whip and tongue variation, a notch is cut downwards into the sliced face of the rootstock and a similar cut upwards into the face of the scion cut. These act as the tongues and it requires some skill to make the cuts so that the scion and the rootstock marry up neatly. The elongated ‘Z’ shape adds strength, removing the need for a companion rod in the first season. The joint is then taped around and treated with tree-sealing compound or grafting wax. A whip graft without a tongue is less stable and may need added support.

Stub grafting is a technique that requires less rootstock than cleft grafting, and retains the shape of a plant. Also scions are generally of 6-8 buds in this process. An incision is made into the branch 1 cm long, then the scion is wedged and forced into the branch. The scion should be at an angle of at most 350 to the parent plant so that the crotch remains strong. The graft is covered with grafting compound. After the graft has taken, the branch is removed and treated a few centimeters above the graft, to be fully removed when the graft is strong.

The four-flap graft (also called banana graft) is heralded for maximum cambium overlap, but is a complex graft. It requires similarly sized diameters for the rootstock and scion. The bark of the rootstock is sliced and peeled back in four flaps, and the hardwood is removed, looking somewhat like a peeled banana.

Awl grafting takes the least resources and the least time. Awl grafting can be done by using a screwdriver to make a slit in the bark, not penetrating the cambium layer completely. Then inset the wedged scion into the incision.

Veneer grafting, or inlay grafting, is a method used for stems larger than 3 cm in diameter. The scion is recommended to be about as thick as a pencil. Clefts are made of the same size as the scion on the side of the branch, not on top. The scion end is shaped as a wedge, inserted, and wrapped with tape to the scaffolding branches to give it more strength.

Rind grafting involves grafting a small scion onto the end of a thick stem. The thick stock is sawn off, and a approximately 4 cm long bark-deep cut is made parallel to the stem, from the sawn-off end down, and the bark is separated from the wood on one or both sides. The scion is shaped as a wedge, exposing cambium on both sides, and is pushed in under the back of the stock, with a flat side against the wood.

The present raspberry plant can be used for improving plant vigour, higher fruit yield, more and better resistances to diseases and the absence or prevention of root suckers during cultivation.

### BRIEF DESCRIPTION OF THE DRAWINGS

The present invention will be further illustrated in the following examples. In the examples reference is made to figures wherein:

FIG. 1: shows the number of laterals per cane. The dark colour indicates the number of laterals per cane, the light colour indicates the number of non-developed buds per cane;

FIG. 2: shows the number of flowers per cane;

FIG. 3: shows fruit(s) of Sapphire (A), Sapphire grafted on TP4 (B) and Sapphire grafted on Diamond (C);

FIG. 4: shows average fruit weight;

FIG. 5: shows the number of fruits and flowers per lateral. The darkest colour indicates the number of harvested fruits per lateral, the middle colour indicates the number of developing fruits per lateral, the lightest colour indicates the number of flowers per lateral;

FIG. 6: the number of fruits and flowers per cane. The darkest colour indicates the number of harvested fruits per cane, the middle colour indicates the number of developing fruits per cane, the lightest colour indicates the number of flowers per cane;

FIG. 7: shows cumulative fruit production per cane;

FIG. 8: shows cumulative number of fruits per cane;

FIG. 9: shows average fruit weight;

### DESCRIPTION OF THE INVENTION

### Examples: Improved Raspberry Plants by Grafting

### Example 1

### Introduction

A general observation for several raspberry cultivars is their vulnerable root system. It is sensitive for different root diseases and dies easily if the water gift is too less or too much during cane development. Grafting of raspberry on a blackberry rootstock leads to a stronger and better developed root system which is more resistant to several root pathogens and can better withstand deviating watering during cane development. Sapphire was grafted on two different rootstocks; the raspberry cultivar Diamond and the blackberry cultivar TP4.

### Method and Results

Sapphire was grafted on two different rootstocks; the raspberry cultivar Diamond and the blackberry cultivar TP4. The development of the grafted canes took place at a nursery field for seven months. Afterwards, the grafted canes were stored in the dark at −1° C. for 3 months. Thereafter, the grafted plants were grown and started to produce fruits in a production field. The grafted plants were observed during cane development and fruit production.

A first observation for the grafted Sapphire plants on Diamond and TP4 was their ability to better withstand deviating watering during cane development. The grafted plants were planted in pots containing one plant, while the other plants in the nursery field were planted in pots containing two plants. So the grafted plants received a double amount of water compared to the standard circumstances. Despite this double amount of water, no adverse effects on cane development were visible. Therefore, the roots of the grafted Sapphire plants on Diamond and TP4 can better withstand deviating watering.

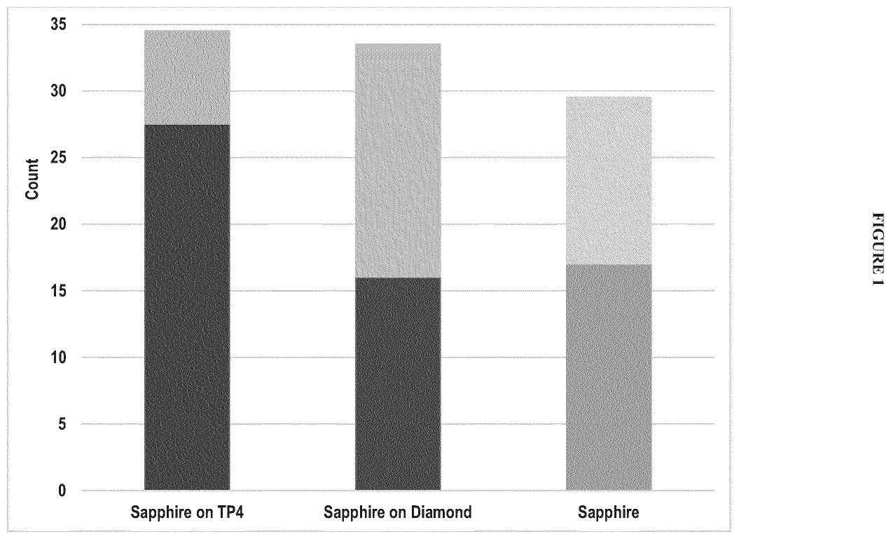

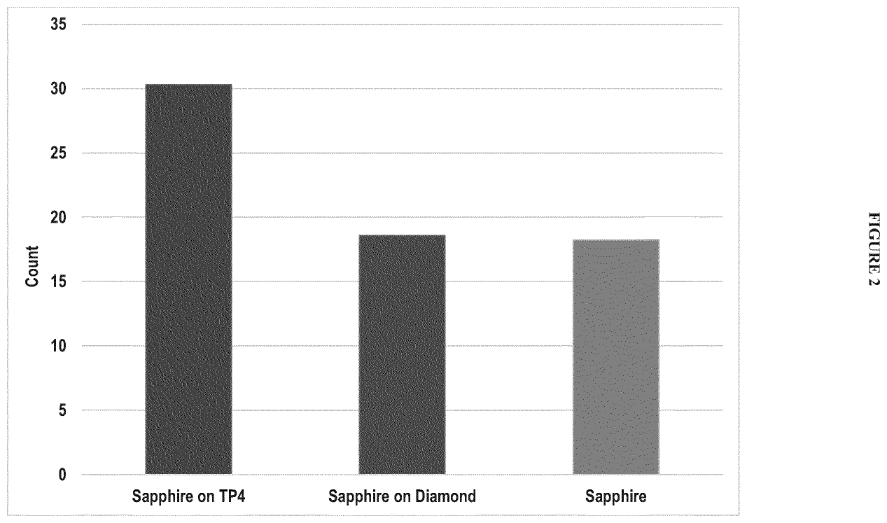

A second observation for the grafted Sapphire plants on TP4 was the increased vigour of the plants during fruit production. The grafted Sapphire plants on TP4 showed an increased number of laterals per cane (FIG. 1) and flowers per lateral (FIG. 2). Next to this, the leaves of the grafted Sapphire plants on TP4 kept a healthy green colour, while the leaves of the non-grafted Sapphire plants showed yellowing of the leaves during fruit production. The plant architecture of the grafted Sapphire plants on Diamond showed no differences compared to the non-grafted Sapphire plants.

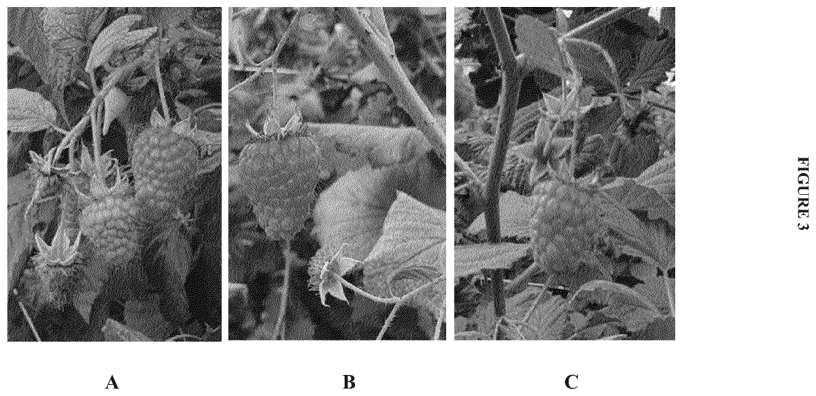

A third observation for the grafted Sapphire plants on TP4 was the increased fruit production. The fruits of the grafted Sapphire plants on TP4 were of similar size and quality as compared with the fruits of the non-grafted Sapphire plants (FIG. 3A-B). The fruits of the grafted Sapphire plants on Diamond were unregular due to uneven droplets (FIG. 3C).

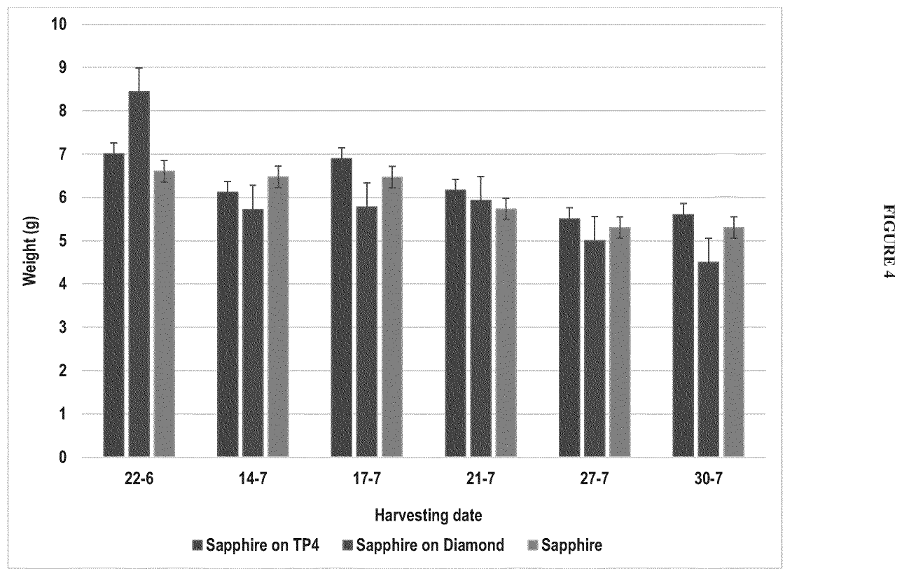

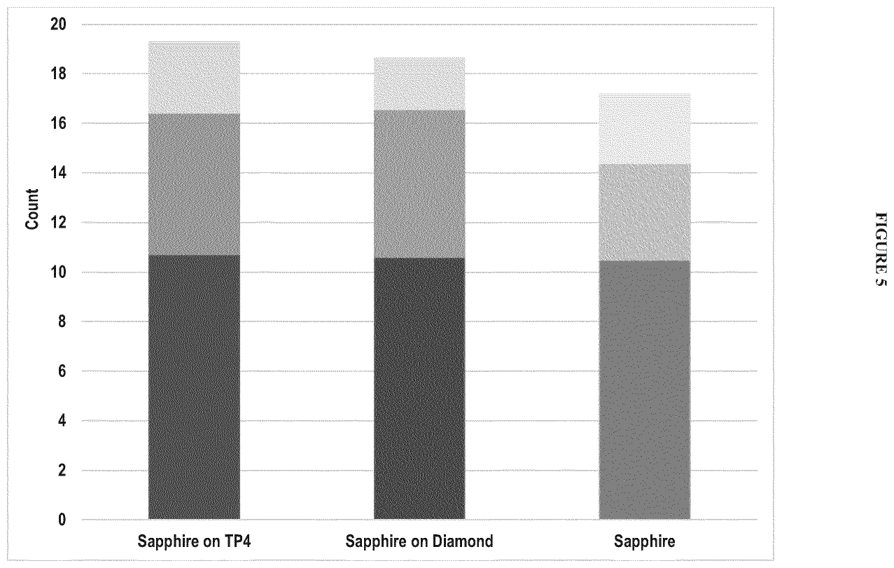

The average fruit weight of the grafted Sapphire plants on TP4 was slightly higher than the average fruit weight of the non-grafted Sapphire plants (FIG. 4). While the average fruit weight of the grafted Sapphire plants on Diamond was slightly lower than the average fruit weight of the non-grafted Sapphire plants. After the harvesting period, the total fruit production was estimated by counting the number of harvested fruits per lateral, the number of fruits in developing phase per lateral and the number of flowers per lateral. The number of laterals per cane was clearly higher for grafted Sapphire plants on TP4 compared to non-grafted Sapphire plants (FIG. 1) while the number of fruits per lateral was comparable (FIG. 5).

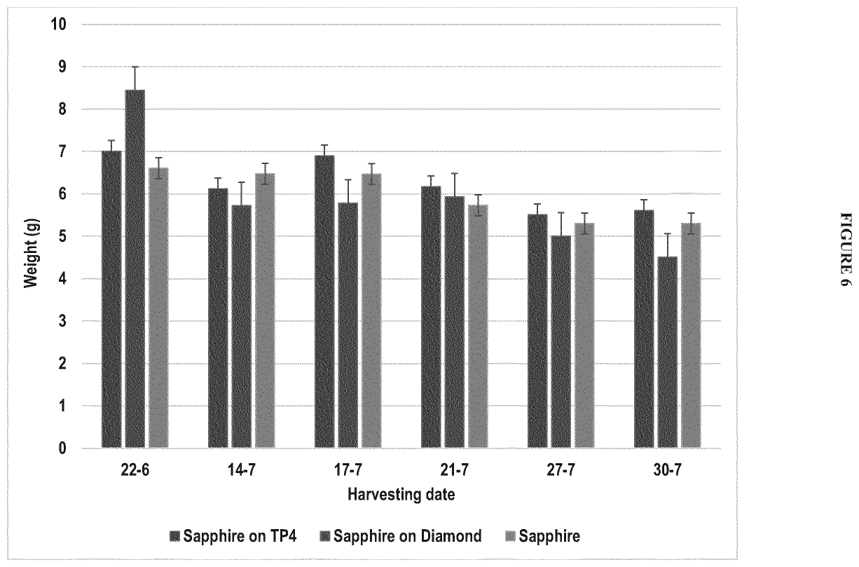

Therefore the number of fruits per cane was higher for grafted Sapphire plants on TP4 compared to the non-grafted Sapphire plants (FIG. 6). This resulted in an increased fruit production for the grafted Sapphire plants on TP4. The fruit production of the grafted Sapphire plants on Diamond showed no differences compared to the non-grafted Sapphire plants.

The last observation for the grafted Sapphire plants on TP4 was the absence of root suckers. The grafted Sapphire plants on Diamond and the non-grafted Sapphire plants produced root suckers. These need to be removed during the fruit production period. The absence of root suckers at the grafted Sapphire plants on TP4 resulted in reduced labour and lower production costs.

### Conclusion

From this example it can be concluded that Sapphire grafted on TP4 provides important improvements in terms of a higher number of laterals per cane, healthy and green leaves, a comparable number of fruits per lateral, a slightly higher average fruit weight and the absence of root suckers.

### Example 2

To check the reliability of the previous described results another experiment was performed i.e. with focus on fruit production. Sapphire was grafted on the blackberry cultivar TP4. The development of the grafted canes took place at a nursery field for six months. Afterwards, the grafted canes were stored in the dark at −1° C. for 2 months. Thereafter, the grafted plants were grown and started to produce fruits in a production greenhouse. The grafted plants were observed during cane development and fruit production.

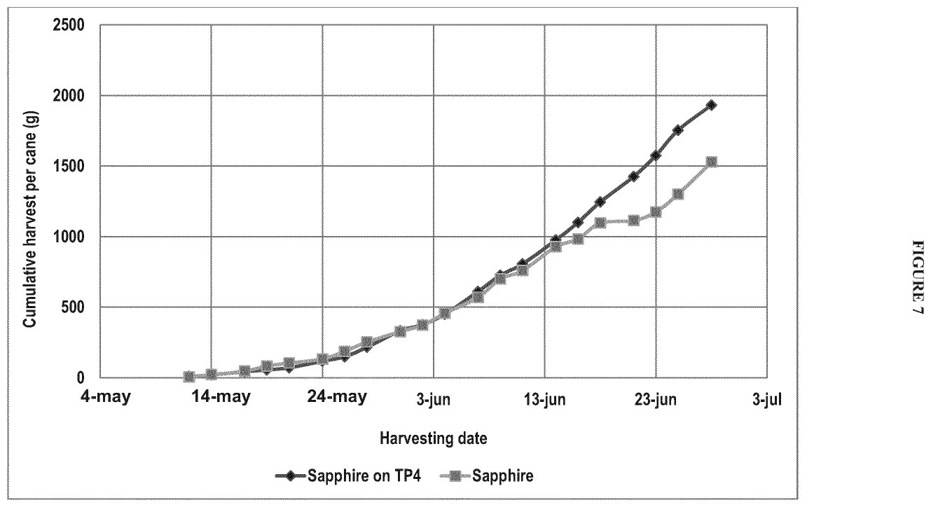

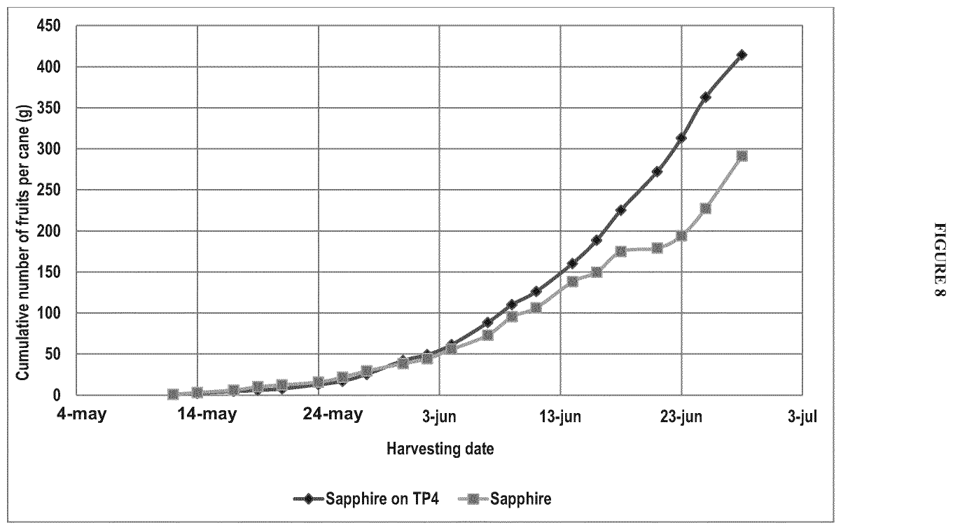

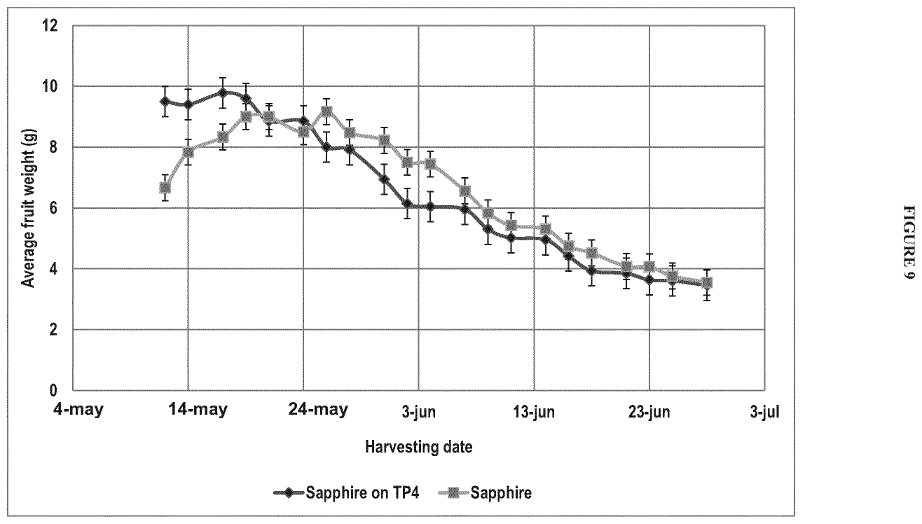

In this Example 2 fruit production of grafted Sapphire plants on TP4 was studied in further detail. The harvest of grafted Sapphire plants on TP4 and the non-grafted plants was comparable in the beginning of the harvesting period (FIG. 7). The harvest of the grafted Sapphire plants on TP4 increased faster than the non-grafted Sapphire plants after three weeks of harvesting. The same pattern was shown for the number of harvested fruits per cane (FIG. 8). The average fruit weight of the grafted Sapphire plants on TP4 was higher than the average fruit weight of the non-grafted Sapphire plants in the beginning of the harvesting period (FIG. 9). The average fruit weight of the grafted Sapphire plants on TP4 and the non-grafted Sapphire plants was comparable at the end of harvesting. These differences resulted in an increased fruit production for the grafted Sapphire plants on TP4 compared to the non-grafted Sapphire plants.

1. A grafted raspberry plant comprising a raspberry scion grafted on a blackberry rootstock.

2. The grafted raspberry plant according to claim 1, wherein the blackberry rootstock is selected from the group consisting of Rubus ursinus, Rubus laciniatus, Rubus argutus, Rubus armeniacus, Rubus plicatus, Rubus ulmifolius, Rubus allegheniensis, Rubus caesius, Rubus gratius, Rubus odoratus, Rubus spectabilis, Rubus stereobelus, Rubus corylifolii spec. (x dumetorum), Rubus sulcatus, Rubus foliosus, Rubus prei, Rubus pedemontanus, Rubus rudis, Rubus oreades, Rubus rosaceus, Rubus derasifolius, Rubus flexuosus, Rubus gratus, Rubus dejonghii, Rubus nemorosus, Rubus glareosus, Rubus geniculatus, Rubus diversus, Rubus lesdainii, Rubus drymophilus, Rubus polyanthemus, Rubus vestitus, Rubus rufescens, Rubus holerythos, Rubus macrophyllus, Rubus ulmifolius, Rubus armeniacus, Rubus nitidiformis, Rubus nemoralis laciniatus, Rubus insectifolius, Rubus subinermoides, Rubus platyacanthus, Rubus vigorosus, Rubus nessensis, Rubus praticolor, Rubus arcticus, and intra specific and inter specific hybrids thereof.

3. The grafted raspberry plant according to claim 1, wherein the raspberry is Rubus idaeus or a hybrid thereof.

4. The grafted raspberry plant according to claim 1, wherein the raspberry is selected from the group consisting of Rubus crataegifolius, Rubus gunnianus, Rubus idaeus, Rubus leucodermis, Rubus occidentalis, Rubus parvifolius, Rubus phoenicolasius, Rubus rosifolius, Rubus strigosus, Rubus ellipticus, and intra specific and inter specific hybrids thereof.

5. A method for providing a grafted raspberry plant, comprising:
a) providing a blackberry rootstock;
b) grafting a raspberry scion on the blackberry rootstock;
c) allowing the graft to develop into a raspberry plant.

6. The method according to claim 5, wherein grafting comprises cleft grafting, whip grafting, stub grafting, four-flap grafting, awl grafting, veneer grafting or rind grafting.

7. The grafted raspberry plant according to claim 1, wherein the grafted raspberry plant exhibits greater fruit yield than an ungrafted raspberry plant.

8. The grafted raspberry plant according to claim 1, wherein the grafted raspberry plant exhibits greater disease resistance than an ungrafted raspberry plant.

9. The grafted raspberry plant according to claim 1, wherein the grafted raspberry plant exhibits greater vigour than an ungrafted raspberry plant.

10. The grafted raspberry plant according to claim 1, wherein the grafted raspberry plant does not produce root suckers during cultivation.

11. The method according to claim 5, wherein the blackberry rootstock is selected from the group consisting of Rubus ursinus, Rubus laciniatus, Rubus argutus, Rubus armeniacus, Rubus plicatus, Rubus ulmifolius, Rubus allegheniensis, Rubus caesius, Rubus gratius, Rubus odoratus, Rubus spectabilis, Rubus stereobelus, Rubus corylifoilii spec. (x dumetorum), Rubus sulcatus, Rubus foliosus, Rubus prei, Rubus pedemontanus, Rubus rudis, Rubus oreades, Rubus rosaceus, Rubus derasifolius, Rubus flexuosus, Rubus gratus, Rubus dejonghii, Rubus nemorosus, Rubus glareosus, Rubus geniculatus, Rubus diversus, Rubus lesdainii, Rubus drymophilus, Rubus polyanthemus, Rubus vestitus, Rubus rufescens, Rubus holerythos, Rubus macrophyllus, Rubus ulmifolius, Rubus armeniacus, Rubus nitidiformis, Rubus nemoralis laciniatus, Rubus insectifolius, Rubus subinermoides, Rubus platyacanthus, Rubus vigorosus, Rubus nessensis, Rubus praticolor, Rubus arcticus, and intra specific and inter specific hybrids thereof.

12. The method according to claim 5, wherein the raspberry is Rubus idaeus or a hybrid thereof.

13. The method according to claim 5, wherein the raspberry is selected from the group consisting of Rubus crataegifolius, Rubus gunnianus, Rubus idaeus, Rubus leucodermis, Rubus occidentalis, Rubus parvifolius, Rubus phoenicolasius, Rubus rosifolius, Rubus strigosus, Rubus ellipticus, and intra specific and inter specific hybrids thereof.
